(19) United States
(12) Patent Application Publication
(10) Pub. No.: US 2009/0269398 A1
(43) Pub. Date: Oct. 29, 2009

(54) Compositions for the encapsulation of natural product extracts in oil medium in hard gelatin capsules and a method of encapsulation

(76) Inventors: Vilambi NRK Reddy, (US); Anil Torgalkar, (US)

Correspondence Address: BREGEN TECHNICAL CONSULTANTS L.L.C., 154 OLD CLINTON ROAD, FLEMINGTON, NJ, 08822, US

(21) Appl. No.: 12/150,192

(22) Filed: Apr. 26, 2008

(51) Int. Cl. A61K 9/48; A61K 47/44; A61K 47/48; A61K 47/36; A61K 47/38; A61K 47/42; A61K 36/889; A61K 36/28; A61K 36/899; A61P 17/06

(52) U.S. Cl. 424/452; 514/783; 514/772; 514/778; 514/773; 514/781; 424/455; 424/727; 424/764; 424/750

(57) ABSTRACT

The present invention relates to compositions of natural extracts in oil medium, prepared in non-free-flowing formulations, and encapsuled in hard gelatin capsules, without post-fill sealing. These compositions comprise a therapeutically effective amount of at least one natural extract in oil medium, the composition suitable for treating a medical condition, and at least one oil-absorbent ingredient in powder form. More specifically, the natural extract in the composition can be an extract of Wrightia tinctoria, which is suitable for treating medical conditions, such as, but not limited to, relief from pain, psoriasis, eczema, spondolytis, acne and wound care. The invention also provides a method for encapsulating the natural extracts in oil medium compositions, compounded in a non-free-flowing formulation, in hard gelatin capsules without the need for post-fill sealing.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISK APPENDIX

Not applicable.

### FIELD OF THE INVENTION

The present invention relates to a method for encapsulating natural product oil extracts in hard gelatin capsules and the compositions suitable for the encapsulation. In particular, the present invention relates to encapsulating an herbal oil in a hard gelatin capsule, the composition and capsule being suitable for oral consumption. Preferably, the oil comprises at least one herbal ingredient obtained from the Wrightia tinctoria plant.

### BRIEF DESCRIPTION OF THE BACKGROUND ART

Natural product extracts in oil medium have long been used for healthcare in Indian traditional medicine.
Murugesa Mudalair, Siddha Materia Medica, Second Edition, 2006, Published by Government Indian Medical School, Chennai 600106, India
The Ayurvedic Formulary of India, Part I, Second Edition. Delhi, Controller of Publications, 2003, ISBN 81-9011514-6
The Ayurvedic Formulary of India, Part II. Delhi, The Controller of Publications, 2000, ISBN 81-901151-1-1; Advances in Ayurvedic Medicine/R. H. Singh. Varanasi, Chaukhambha Visvabharati, 2005, 5 volumes

The natural product extracts can be obtained from plant or animal sources. For external (topical) use, these natural product extracts in oil medium have been formulated primarily into oils, creams or ointments. Those that are intended for internal use have been predominantly encapsulated in soft gel capsules and taken orally (eg. Fish oil, flax seed oil, etc);
Murray, Michael T., Encyclopedia of Nutritional Supplementation, Prima Publishing, Rocklin, Calif., 1996.
Simopoulos, Artemis P., Robinson, Jo, The Omega Plan, HarperCollins Publishers, New York, 1998.

Soft gel encapsulated products suffer from several disadvantages [Ewart T. Cole, Liquid filled and sealed hard gelatin capsules, Gattefosse Bulletin nr 92 (1999)] both in the manufacturing process and in the encapsulated product as well, including: the migration of active ingredients; the high permeability of the soft gel; the fact that primary production usually needs to be done through contract manufacturers, because of a complex and messy operation, thus compromising confidentiality and costs; the high probability of a large dimensional variation of the product, leading to problems in primary packaging; the impracticality of low batch production, because of the huge and complex changeover required for each product; and the poor aesthetics of the soft gel products.

The hard gelatin capsule, conventionally used as a dosage form for Rx and OTC drugs and herbal products, offsets most of the disadvantages discussed above; but is designed to be used only with ingredients formulated either as powder or pellets.

Research and development efforts are attempting to enable the hard gel capsules to be filled with liquid formulations, a process requiring the need for post-fill sealing steps to ensure that the liquid does not leak in storage (syneresis.) An excellent review of the “Liquid filled and sealed hard gelatin capsule” technology and the criteria for the choice of materials and formulation guidelines are presented in Ewart's article.

Post-fill sealing causes its own set of problems in manufacture: The method is expensive and cumbersome and the machines are complicated and unreliable, making it very difficult to guarantee that post-fill sealing is leak-free.

Objects of this invention provide compositions comprising natural product extracts in an oil medium and a method for encapsulating the natural product extracts in oil medium in hard gelatin capsules with no post-sealing steps required. Specifically, the present invention provides compositions related to encapsulating an herbal oil extract in a hard gelatin capsule, the formulation and capsule being suitable for oral consumption. The capsule prepared according to this invention preferably contains at least one herbal ingredient obtained from the Wrightia tinctoria plant. The invention also provides a method for preparing these capsules, a method which does not require post-fill sealing. In addition, the invention provides a method of treating medical conditions using the encapsulated compositions of this invention.

### SUMMARY OF THE INVENTION

The present invention relates to compositions of natural product extracts in oil medium, prepared in non-free-flowing (semi-solid) formulations, and then encapsuled in hard gelatin capsules, without the need for a post-fill sealing step. The compositions are blends or uniform mixtures of oil extracts and powdered ingredients. The compositions comprise a therapeutically effective amount of at least one natural product extract in oil medium, the composition suitable for treating a medical condition, and at least one oil-absorbent ingredient in powder form. The compositions may also comprise therapeutically effective amounts of other active natural substances or products and/or pharmaceutically acceptable excipients in oil or powder form. More specifically, the natural product extract in the composition can be an extract of Wrightia tinctoria, which is suitable for treating medical conditions, such as, but not limited to, relief from pain, psoriasis, eczema, spondolytis, acne and wound care, when the preparation is given to a patient in need of the medication.

The invention also provides a method for encapsulating the natural product extracts in oil medium compositions, in a non-free-flowing formulation, in hard gelatin capsules without the need for post-fill sealing.

### BRIEF DESCRIPTION OF THE DRAWINGS

FIG. 1: A flow chart depicting one example of the encapsulation method

### DETAILED DESCRIPTION OF THE INVENTION

It is an object of the present invention to provide a composition comprising a natural product in an oil medium, the composition which is compounded or blended into a non-free-flowing formulation suitable for encapsulation in hard gelatin (gel) capsules without a post-fill sealing step.

It is another object of the invention to provide a method for encapsulating the non-free-flowing formulation, comprising one or more natural product extracts in an oil medium, in hard gelatin capsules. The capsules do not require post-fill sealing to prevent syneresis. The hard gelatin capsules are filled, closed and snap-fit sealed in a manner conventionally used with powder or pellet formulations.

The method described herein offers a viable option to formulate semi-solid compositions in hard gelatin capsules. Using this method, the machines are low cost, the process is robust and forgiving, reliability is high, yields are very high, approaching 100%, and the process can be used with very small batch sizes.

More specifically, it is an object of the invention to provide compositions comprising a therapeutically effective amount of at least one herbal ingredient from Wrightia tinctoria, which is further compounded or blended with other active natural products and/or excipients into a non-free-flowing formulation. It is another object of the invention to provide a method for encapsulated the non-free-flowing formulations in hard gelatin capsules with no need for post-fill sealing. The capsules thus prepared and taken orally by a mammal, especially a human, who is in need of the treatment, the capsules of which comprise a therapeutically effective amount of Wrightia tinctoria, and, optionally, other ingredients. The formulations enclosed in said capsules can be useful in the management of medical conditions, such as, but not limited to, psoriasis.

In recent times, natural products are finding increased use in healthcare treatments. The primary sources of natural materials used in healthcare are of plant or herbal origin. Many of these natural materials can be extracted in oil medium and delivered transdermally, for example, for topical and systemic use [Agasthiyar Vaithya Suthram—650, S. S. Mathrubootheswaran, First edition, December 2005, Published by Narmadha Pathipagam, Chennai, India]. Some examples include herbal oil preparations for relief from pain, psoriasis, eczema, spondylitis, acne and wound care.

The oil medium of the present invention for the herbal extract is a non-volatile oil, wherein the non-volatile oil is preferably a vegetable oil such as coconut oil (Cocos nucifera), gingelly oil (sesame oil), sunflower oil, corn oil, or refined vegetable oil. The non-volatile oil in the extract of the present invention comprises from about 80% to 99% by weight of the extract.

One of the natural product extracts in oil medium that can be compounded or blended in non-free-flowing formulations (semi-solids) and encapsulated in hard gel capsules without the post-fill sealing step is derived from Wrightia tinctoria and can be prepared from either the leaves, twigs, flowers, fruit, or stems of the Wrightia tinctoria plant or a combination of these parts of the plant. Wrightia tinctoria is an apocynaceae tree growing throughout India. Its flowers are white and fragrant. When the natural product extract in oil is Wrightia tinctoria, and is present in the capsules in a therapeutically effective amount, the formulation is suitable for the management of medical conditions, exemplified but not limited to, psoriasis.

The oil extract of Wrightia tinctoria or another natural ingredient can be prepared at ambient temperature by any suitable process known to those skilled in the art. For example, it can be prepared by the process disclosed in Ser. No. 12/009,799 filed on Jan. 22, 2008, a continuation-in-part of Ser. No. 11/258,923 filed on Nov. 28, 2005. This process is disclosed below.

The non-aqueous extract of Wrightia tinctoria is prepared at ambient temperature by cleaning and pulverizing the selected parts of the Wrightia tinctoria plant and soaking them in a non-aqueous oil medium containing coconut oil. Care should be taken to add sufficient oil medium to ensure that the plant material is completely submerged. The plant material/oil medium is then irradiated with a light source in the spectrum range of 300-1100 nm for a period of approximately 5 days. During this time the herbal ingredients are allowed to extract into the non-aqueous oil medium. At the end of the extraction, the oil medium is a purplish brown color. It is then filtered and the filtrate is stored for further processing as the non-aqueous herbal extract of Wrightia tinctoria. The non-aqueous extracts of Wrightia tinctoria are compounded with the other ingredients mentioned herein in preparing the non-free-flowing formulations to fill hard gelatin capsules without post-fill sealing.

Other herbal extracts in the formulation may include Melia azadirachta Linn oils (Neem), which, when used in therapeutically effective amounts to those who need it, are documented to have beneficial skin effects:
Chopra, R. N., Nayar, S. C., and Chopra I. C., Glossary of Indian Medicinal Plants, C.S.I.R., P.259, 1956.
Nair, C. P. R., Kurup, P. B., Pillai, K. G. B., Geetha, A., and Ramiah, N., Effect of Nimbidin in Psoriasis, Indian Medical Journal, October 1978.
   The composition of the natural product extracts in oil medium of the present invention may comprise an extract of at least one other active herbal ingredient, such as those mentioned above, in the amount of from 1% to 20% by weight of the extraction medium.

Compositions of the present invention for encapsulating natural product extracts in oil medium in non-free-flowing formulations in hard gel capsules by a process not requiring a post-fill sealing step include at least one oil absorbent ingredient in powder form. The oil absorbent ingredient is preferentially an edible powder from synthetic or natural sources having the property of providing significant absorption of the oil into the absorbent. The range considered to be significant is 5 to 40% w/w of oil absorption in powder.

The powder may absorb the oil by any known method, or combination of methods, two of which are identified here: physical absorption or the absorption by means of the microporous or nanoporous structure of the powder, a process controlled by the particle size and the morphology of the powder. Conceivably, the oil can be adsorbed by a powder, either in conjunction with absorption or alone, and form an effective semi-solid or non-free-flowing formulation, suitable for encapsulation in an embodiment of the invention.

Suitable candidates for the edible oil absorbent ingredient from natural sources preferably include powdered herbal medicinal plants, food grains, vegetables, fruits, nuts, milk, starch and sugar. Some of the preferred herbal medicinal powders of the present invention for use as an oil absorbent ingredient include a mixture of the powder of the dried fruits of Terminalia chebula, Terminalia belerica, and Emblica officinalis. The mixture of Terminalia chebula, Terminalia belerica, and Emblica officinalis, called Triphala, is not only effective as an oil absorbent powder but has been found to be very effective in helping to control weight gain, chronic constipation and as an adjunctive treatment for many chronic degenerative conditions. Triphala also offers a range of additional health benefits including, but not limited to: improving digestion, reducing serum cholesterol, improving circulation (potentiating the adrenergic function), exerting a marked cardio-protective effect, reducing high blood pressure, improving liver function, has proven anti-inflammatory and anti-viral properties, acts as an expectorant, a hypotensive, anticarcinogenic and antioxidant agent, and contains 31% linoleic acid. [The wonders of Triphala: Ayurvedic Formula for Internal Purification, by Dr. Michael Tierra, L. Ac., O.M.D 1966; Nakanishi, K., Chem. Pharm. Bull., (Tokyo), 13 (1965), 882].

Compositions of the invention for encapsulating non-free-flowing formulations of natural product extracts in oil medium in hard gel capsules, the capsules filled without the need for post-fill sealing, may also include other edible herbal ingredients in powder form and may preferably include powdered Zingiber officinale (ginger.)

The composition of this invention may also include one or more pharmaceutically-acceptable excipients such as binders, fillers, emulsifying agents, plasticizers, flavoring agents, coloring agents and preservatives as needed. The composition of the present invention for encapsulating natural product extracts in oil medium prepared in non-free-flowing formulations for packing in hard gelatin capsules without the need for post-fill sealing generally comprises oil-absorbent powder ingredients, other active ingredients and other pharmaceutically accepted excipients such as binders, fillers, emulsifying agents, plasticizers, flavoring agents, coloring agents and preservatives, such as Neem, in the amount from 80 to 99 weight percent.

The oil-absorbent powder ingredients may be selected from either natural or synthetic sources. The powdered edible oil-absorbent ingredient from natural sources is selected from the group: herbal medicinal plants, food grains, vegetables, fruits, nuts, milk, starch and sugar. The powdered edible oil-absorbent ingredient from synthetic sources is selected from the group: synthetic food substitute products, polysaccarides, proteins, and chemically-modified cellulose.

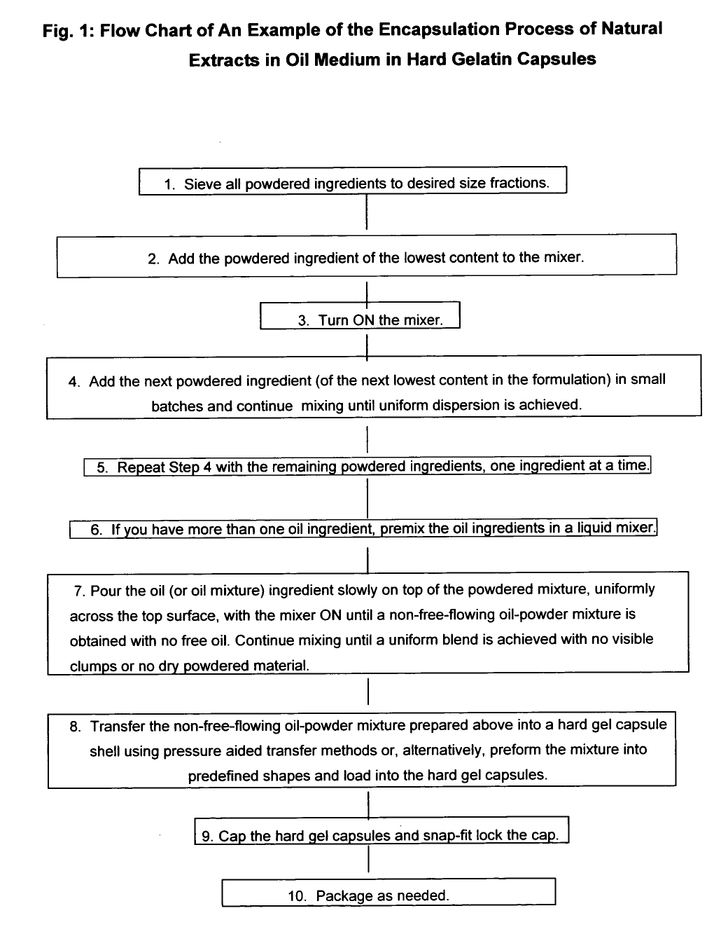

A method of preparing the hard gel capsules described in the present invention, that is, those comprising therapeutically effective amounts of natural product extracts in oil medium, compounded or blended into non-free-flowing formulations, the method which does not require a step for post-fill sealing, may include blending of the product extract(s) in oil medium with oil absorbent ingredient(s) in powder form and, optionally, one or more other active natural ingredients/pharmaceutically-acceptable excipients. This method or process can form a non-free-flowing mixture with no syneresis of the oil. The hard gel capsules are then filled with the non-free-flowing mixture (semi-solid) prepared above and capped to produce the encapsulated product. It is preferred that the blending of the oil and powder components of the present invention be done at low speed, low shear and at low temperatures to produce a non-free-flowing mass of the oil extract and powder mixture. It is also preferred that the filling of the hard gel capsules with the non-free-flowing mixture of the oil extract and powder ingredients discussed above be done with no post-fill-sealing by pressure transfer directly into the hard gel capsule or by shaping with a die into small cylinders of the size to fit inside the hard gel capsule. A flow chart comprising the steps of an example of this method are included in FIG. 1.

An example of a composition which can be compounded or blended into a non-free-flowing formulation as an object of this invention is presented in Table 1 and a method for encapsulating the natural product extracts in oil medium in non-free-flowing formulations in hard gel capsules without the need for post-fill sealing follows:

### EXAMPLE 1

TABLE 1

| Main Ingredients | wt (gms) | wt %* |
|---|---|---|
| Wrightia tinctoria (oil extract) | 75 | 15 |
| Terminalia chebula (powder) | 125 | 25 |
| Terminalia belerica (powder) | 125 | 25 |
| Emblica officinalis (powder) | 125 | 25 |
| Ginger (powder) | 25 | 5 |
| Neem leaves (powder) | 25 | 5 |

*The weight percent of the ingredients in this example is accurate, however, small amounts of other ingredients may be included in the formulations, changing the wt % of the main ingredients.

Add 25 gms of Neem powder to 25 gms of ginger powder and mix until the consistency of the dispersion is uniform. Any standard powder mixer with slow speed and low shear (like a horizontal sigma blade mixer) can be used. Then add 125 gms of Emblica officinalis powder to the mixture and continue mixing until the consistency of the dispersion is uniform. Then add 125 gms of Terminalia belerica powder to the mixture and continue mixing until the consistency of the dispersion is uniform. Then add 125 gms of Terminalia chebula powder to the mixture and continue mixing until the consistency of the dispersion is uniform. When all the powders are well mixed, add 75 gms of Wrightia tinctoria oil extract slowly across the top surface of the powder mixture and continue mixing until all the oil is uniformly absorbed into the powder mixture. If the oil content in the formulation is too low, mix the oil in a solvent that can be dried out after the mixing, leaving the oil uniformly distributed in the powder mixture. The resulting oil-powder formulation will be a non-free-flowing mixture with no syneresis of the oil. Transfer the non-free-flowing oil-powder mixture formulation into the hard gel capsule by direct transfer and apply pressure to compact the oil-powder mixture into the hard gel capsule shell. When the bottom part of the capsule is filled, cap the hard gel capsule by sliding the top part over the bottom part and snap fitting the capsule to secure the closure.

Hard gel capsules prepared as described above were put on stability at 40 degrees C. and followed up at intervals up to 6 months. It was found that no separation of the oil from the oil-powder mixture occurred and no visible leaks in the capsule could be seen. This stability is equivalent to 2 years stability at 25 degrees C.

It is clear from the example above that therapeutically effective amounts of compositions of natural product or herbal extracts in oil medium can be mixed with oil absorbing ingredients in powder form and other natural active ingredients and/or pharmaceutically acceptable excipients to produce a non-free-flowing oil-powder formulation, which can be packed into hard gel capsule shells using pressure aided transfer methods or preformed into predefined shapes and loaded into the hard gel capsules and capped to be packed. It will be apparent to those skilled in the art that other filling methods may also be used and that these methods also fall within the scope of the invention.

It is also clear that hard gel capsules for the management of psoriasis can be prepared with the compositions and methods of the present invention using therapeutically effective amounts of the oil extract of Wrightia tinctoria and mixtures of powders of Emblica officinalis, Terminalia belerica, Terminalia chebula, ginger and Neem. The hard gelatin capsules so prepared do not require post-fill sealing to prevent syneresis.

Other modifications and variations of the present invention will become apparent to those skilled in the art from an examination of the above specification and examples. Therefore, other variations of the present invention may be made which fall within the scope of the appended claims even though such variations were not specifically discussed above.

I claim:

1. A semi-solid composition for encapsulation in hard gelatin capsules without the need for a post-fill sealing step, the composition comprising:
at least one natural product extract in an oil medium,
at least one edible oil-absorbent ingredient in powder form,
and, optionally, at least one pharmaceutically acceptable excipient in oil or powder form,
the ingredients blended together to form a uniform dispersion.

2. The composition according to claim 1, wherein the natural product extract in an oil medium is obtained from plant or animal sources.

3. The composition according to claim 1, wherein the oil medium for the natural product extract is a non-volatile oil, selected from the group: coconut oil (Cocus nucifera), gingelly oil (sesame oil), sunflower oil, soy oil, corn oil and refined vegetable oil.

4. The composition according to claim 3, wherein the non-volatile oil is present in the extract in the amount of from 80% to 99% by weight of the extract.

5. The composition according to claim 1, wherein the oil absorbent ingredient is an edible powder, obtained from synthetic or natural sources.

6. The composition according to claim 5, wherein the powdered edible oil-absorbent ingredient obtained from natural sources is selected from the group: herbal medicinal plants, food grains, vegetables, fruits, nuts, milk, starch and sugar.

7. The composition according to claim 5, wherein the powdered edible oil-absorbent ingredient obtained from synthetic sources is selected from the group: synthetic food substitute products, polysaccharides, proteins, and chemically-modified cellulose.

8. The composition according to claim 7, wherein the chemically-modified cellulose is hydroxypropylmethyl cellulose or carboxymethyl cellulose.

9. The composition according to claim 5, wherein the ratio of oil to oil absorbent powder is 5 to 40 weight percent of the mixture.

10. The composition according to claim 5, which, when packed into a hard gelatin capsule shell, and snap-fit locked, shows no visible evidence of syneresis of the oil from the oil absorbent powder for a period of up to 2 years at 25 degrees C.

11. The composition according to claim 1, further comprising at least one additional active ingredient.

12. The composition according to claim 11, wherein the at least one additional active ingredient is an herbal medicinal product in oil or powdered form.

13. The composition according to claim 1, wherein the pharmaceutically acceptable excipients are selected from the group: binders, fillers, emulsifying agents, plasticizers, flavoring agents, coloring agents, and preservatives.

14. A hard gelatin capsule containing a semi-solid composition of natural product extracts in an oil medium, comprising:
a. a therapeutic amount of at least one natural product extract in an oil medium,
b. at least one oil absorbent ingredient in powder form,
c. optionally, at least one pharmaceutically acceptable excipient in oil or powder form
d. optionally, a therapeutically effective amount of at least one other active natural product ingredient in the form of an oil or a powder,
the capsule capped and snap-fit locked and not requiring post-fill sealing.

15. A method for encapsulating the compositions of claim 1, comprising the steps:
a. sieving the powdered ingredients to obtain the desired size fractions
b. adding the powdered ingredient with the lowest content in the formulation to the mixer in small batches
c. continuing to mix until the ingredient is dispersed
d. adding the powdered ingredient with the next lowest content, if any, and mixing until the powder is dispersed
e. repeating step d as often as needed until all powdered ingredients are dispersed in the mixer
f. adding the oil or mixture of oils to the mixer by pouring it slowly to the top of the powdered mixture
g. continuing to mix until the oil and powder are blended into a semi-solid oil-powder mixture
h. transferring the semi-solid mixture into a hard gelatin capsule shell
i. capping the hard gelatin capsules
j. snap-fit locking the capsule

16. The method of claim 15, whereby in the method of transferring the semi-solid mixture to the capsule shell is a pressure added transfer method which is used alone or in combination with other methods of transfer.

17. The method of claim 15, whereby in step h the method of transferring the semi-solid mixture to the capsule shell, comprises pre-forming the mixture into predefined cylindrical shapes of a size that would fit into the capsule shell and loading them into the hard gel capsule shell.

18. The method of claim 15, wherein the blending is done at low speed, low shear and at low temperatures below 60 Deg. C. to produce a non-free-flowing mass of the oil extract and powder mixture.

19. A semi-solid composition for encapsulation in hard gelatin capsules without the need for a post-fill sealing step, the composition comprising:
at least one herbal extract of Wrightia tinctoria in an oil medium,
at least one mixture of an oil-absorbent powder of the dried fruits of Terminalia chebula, Terminalia belerica and Emblica officinalis,
optionally, at least one pharmaceutically acceptable excipient in oil or powder form,
and, optionally, at least one other active natural ingredient in powder or oil form.
the ingredients blended together to form a uniform dispersion.

20. The composition according to claim 19, which, when comprised of a therapeutically effective amount of the active ingredients and when taken orally by a patient in need thereof, is useful for the management of psoriasis.

21. The composition according to claim 19, wherein the oil medium for the herbal extract of Wrightia tinctoria is a non-volatile oil selected from the group: coconut oil (Cocus nucifera), gingelly oil (sesame oil), sunflower oil, soy oil, corn oil and refined vegetable oil.

22. The composition according to claim 19, wherein the non-volatile oil is present in the extract in the amount of from 80% to 99% by weight of the extract.

23. The composition according to claim 19, wherein the mixture of oil absorbent ingredients are comprised of edible powders obtained from the dried fruits of Terminalia chebula, Terminalia belerica and Emblica officinalis, powders which can absorb the oil into the absorbent in the range of from 5 to 40 weight percent of the total oil powder mixture.

24. The composition according to claim 19, wherein the oil absorbent powder from the dried fruit of Terminalia chebula is present in the powdered mixture in the amount of from 25% to 40% by weight of the powder.

25. The composition according to claim 19, wherein the oil absorbent powder from the dried fruit of Terminalia belerica is present in the powdered mixture in the amount of from 25% to 40% by weight of the powder.

26. The composition according to claim 19, wherein the oil absorbent powder from the dried fruit of Emblica officinalis is present in the powdered mixture in the amount of from 25% to 40% by weight of the powder.

27. The composition according to claim 19, wherein an additional active ingredient is powdered ginger.

28. The composition according to claim 27, wherein the powdered ginger is present in the powdered mixture in the amount of from 12% to 20% by weight.

29. The composition according to claim 19 wherein the herbal extract of Wrightia tinctoria is present in the amount of from 5% to 25% by weight.

30. The composition according to claim 19, which, when packed into a hard gelatin capsule shell and capped, shows no visible evidence of syneresis of the oil from the oil absorbent powder for a period of up to 2 years at 25 degrees C.

31. The composition according to claim 19, wherein the pharmaceutically acceptable excipients are selected from the group: binders, fillers, emulsifying agents, plasticizers, flavoring agents, coloring agents, and preservatives.

32. A hard gelatin capsule, the capsule closed and snap-fit locked, suitable for the management of psoriasis when taken orally by a person in need thereof, containing a composition comprising:
a. a therapeutic amount of at least one herbal extract of Wrightia tinctoria in an oil medium,
b. at least one mixture of oil absorbent herbal powders, comprising the dried fruits of Terminalia chebula, Terminalia belerica and Emblica officinalis
c. optionally, at least one pharmaceutically acceptable excipient in oil or powder form
d. a therapeutically effective amount of powdered ginger and, optionally, at least one other active natural product ingredient in the form of an oil or a powder.

33. A method for encapsulating the compositions of claim 19, comprising the steps:
a. sieving the powdered ingredients to obtain the desired size fractions
b. adding the powdered ingredient with the lowest content in the formulation to the mixer in small batches
c. continuing to mix until the powdered ingredient is dispersed
d. adding the powdered ingredient with the next lowest content, if any, and mixing until the powders are dispersed
e. repeating step d as often as needed until all powdered ingredients are dispersed in the mixer
f. adding the oil or mixture of oils to the mixer by pouring it slowly to the top of the powdered mixture
g. continuing to mix until the oil and powder are blended into a semi-solid oil-powder mixture
h. transferring the semi-solid mixture into a hard gelatin capsule shell
i. capping the hard gelatin capsules
j. snap-fit locking the capsule

34. The method of claim 33, whereby in the method of transferring the semi-solid mixture to the capsule shell is a pressure added transfer method, used alone or in combination with other methods.

35. The method of claim 33, whereby in step h the method of transferring the semi-solid mixture to the capsule shell, comprises pre-forming the mixture into predefined cylindrical shapes of a size that would fit into the capsule shell and loading them into the hard gel capsule shell.

36. The method of claim 33, wherein the blending is done at low speed, low shear and at low temperatures below 60 Deg. C. to produce a non-free-flowing mass of the oil extract and powder mixture.

37. A method of treating psoriasis in a human, comprising administering to the human in need thereof a regimen of a therapeutically effective amount of the composition of claim 33.

38. A method of treating psoriasis in a human, comprising administering to the human in need thereof a regimen of a therapeutically effective amount of the composition of claim 33 in the dosage form of a hard gelatin capsule with no need for a post-fill sealing step.

39. A dosage form for treating psoriasis, wherein the hard gelatin capsule requiring no post-fill sealing step contains the semi-solid blend of ingredients of the composition of claim 19.
